(19) United States
(12) Patent Application Publication
(10) Pub. No.: US 2010/0211115 A1
(43) Pub. Date: Aug. 19, 2010

(54) Compression screw assembly, an orthopedic fixation system including a compression screw assembly and method of use

(76) Inventors: Jeff Tyber, Bethlehem, PA (US); Gannoe Jamy, West Milford, NJ (US)

Correspondence Address: WARD & OLIVO, SUITE 300, 382 SPRINGFIELD AVENUE, SUMMIT, NJ, 07901, US

(21) Appl. No.: 12/317,498

(22) Filed: Dec. 24, 2008

(51) Int. Cl. A61B 17/86; A61B 17/58

(52) U.S. Cl. 606/305; 606/309; 606/104

(57) ABSTRACT

A compression screw assembly includes a first primary member having a threaded leading portion of a first pitch, a smooth middle portion and a threaded trailing portion of a second pitch with the trailing portion residing directly opposite leading portion. The compression screw assembly includes a second threaded screw head member positioned at a distal end having external threads, which are of the same pitch as the plurality of threads of the leading portion, and internal threads of a fourth pitch and dissimilar screw leads from the external threads. The difference in screw leads causes the compression screw member to apply a compressive force on bone fragments when compression screw assembly is inserted into bone fragments.

### FIELD OF THE INVENTION

This invention relates to the field of orthopedic implant devices, and more particularly, to a compression screw assembly, an orthopedic fixation system and a method of utilizing the compression screw assembly to provide a compressive force to secure two or more bone fragments or bones together.

### BACKGROUND OF THE INVENTION

Orthopedic fixation systems, which include orthopedic implant devices are often used to repair or reconstruct bones and joints, and to repair bone fractures, degenerative bone conditions and similar types of injuries.

Frequently, these systems require that bone fragments, such as cracked, broken, or osteotomy bones be kept attached together for lengthy periods of time under a sustained force across the fractured site in order to promote healing. As such, these systems serve to apply interfragmental compression to bone fragments as well as to realign bone segments and to restore native geometries.

The orthopedic implant devices used to reconstruct bones are constructed from either one-piece or two-piece compression screw assemblies. A one-piece compression screw assembly is constructed from a single member and has an elongated body that terminates into a threaded screw head. This elongated body, which is threaded, cooperates with the threaded screw head to apply interfragmental compression to bone fragments. Such screw assemblies, not having an independent screw head, enable only a moderate amount of compression to be applied to bone fragments.

On the other hand, a two-piece compression screw assembly is constructed from a threaded screw shank and an independent screw head. The threaded screw shank is threaded onto the screw head to form a unitary compression screw assembly and is inserted into bone to apply interfragmental compression.

The two-piece compression screw assembly provides compression across bone fragments when inserted into those fragments. The screw shank, having external threads of a certain pitch, is coupled to the threaded screw head having external threads of yet another pitch. The pitch differential between the screw shank and the screw head of a two-piece compression screw assembly causes the screw to apply a compressive force against bone fragments when inserted into those fragments. However, this compression screw assembly is uncontrollable because there is no limit or control on the amount of compression applied to the bone fragments. As the threads on the screw shank oppose the compression applied by the screw head when the two-pieces are rotated within the bone fragments, interfragmental compression is weakened. In addition, the screw head may torque beyond the limits that the bone fragment can handle, causing bone trauma and affecting the proper healing of the fracture.

There is therefore a need for a compression screw assembly, system and method of use that overcomes the previously delineated drawbacks of prior compression screw assemblies.

### SUMMARY OF THE INVENTION

An object of the invention is to overcome the above-mentioned drawbacks of previous fixation systems.

Another object of the invention is to provide a novel and useful orthopedic implant device utilizing a compression screw assembly that may be utilized to secure multiple bones fragments or bones together.

Another object of the invention is to provide a compression screw assembly that may be utilized to secure the interfragmental interface.

Another object of the invention is to apply compression to separated bone fragments via an independent screw head.

Another object of the invention is to provide a compression screw driver assembly that is utilized to hold and transmit insertion torque to the compression screw assembly.

Another object of the invention is to provide a compression screw driver assembly that is utilized to provide a controlled application of compression during insertion of the compression screw assembly into bone.

Finally, an object of the invention is to provide a screw driver assembly that is utilized to reposition the tip of the compression screw assembly after compression is achieved.

In a first non-limiting aspect of the invention, an orthopedic fixation system including a compression screw assembly is provided comprising a primary screw member having a threaded leading portion, an opposite threaded trailing portion and a smooth middle portion disposed between the leading portion and the trailing portion. The leading portion has a plurality of first threads having a first pitch. The trailing portion has a plurality of second threads having a second pitch. The compression screw assembly also includes a screw head having a threaded outer surface. The threaded outer surface has a plurality of third threads having a third pitch, whereby the screw head defines a central opening with a threaded inner surface. The threaded inner surface has a plurality of threads having a fourth pitch, wherein the threaded inner surface is adapted for mating engagement on the threaded trailing portion of the primary screw member. Additionally, the first pitch and the third pitch are approximately identical while the second pitch and the fourth pitch are approximately identical. Furthermore, the screw leads of the threaded trailing portion are greater that the second pitch.

In a second non-limiting aspect of the invention, an orthopedic fixation system is provided comprising a compression screw assembly and a compression screw driver assembly.

The compression screw assembly comprises a primary screw member having a threaded leading portion, an opposite threaded trailing portion and a smooth middle portion disposed between the leading portion and the trailing portion. The leading portion has a plurality of first threads having a first pitch. The trailing portion has a plurality of second threads having a second pitch. The compression screw assembly also includes a screw head having a threaded outer surface. The threaded outer surface has a plurality of third threads having a third pitch, whereby the screw head defines a central opening with a threaded inner surface. The threaded inner surface has a plurality of threads having a fourth pitch, wherein the threaded inner surface is adapted for mating engagement on the threaded trailing portion of the primary screw member. Additionally, the first pitch and the third pitch are approximately identical while the second pitch and the fourth pitch are approximately identical. Furthermore, the screw leads of the threaded trailing portion are greater than that of the second pitch.

The compression screw driver assembly is utilized for engaging the compression screw assembly. The compression screw driver assembly comprises a proximal compression shaft member having a first end and an opposed second end. The first end is coupled to a ratchet assembly while the second end receives a pin for controlling the rotation of the screw driver assembly. The compression screw driver assembly also includes a distal compression shaft member. The distal compression shaft member has a third end coupled to the second end of the proximal compression shaft member. Also included is a fourth end for controlling rotational movement of the screw head. The compression screw driver assembly also includes a primary shaft member, which resides within the proximal shaft member and also resides within the distal shaft member. The primary shaft member has an end, which is provided for controlling rotational movement of the primary screw member. Finally, the compression screw driver assembly has a clutch assembly for selectively engaging and controlling the independent rotational movement of the primary screw member and the screw head.

In a third non-limiting aspect of the invention, a method of compressing bone fragments is provided and comprises seven steps. In step one, a compression screw assembly having a primary screw member and a screw head is provided. Next, in step two, a tissue protect guide is placed at an entry location of the compression screw assembly into bone. In step three, a guide wire is inserted into the bone at the entry location. Next, in step four, a hole is drilled at the entry location to a predetermined depth. In step five, the compression screw assembly is coupled to a compression screw driver assembly. Next, in step six, the compression screw driver assembly is inserted over the guide wire and rotated to insert compression screw assembly into bone. The compression screw assembly is inserted by rotating the primary screw member and the screw head. Finally, in step seven, the screw head is further rotated while preventing the primary screw member from rotating to compress bone fragments.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of an orthopedic fixation system comprising a compression screw assembly and a compression screw driver assembly according to the preferred embodiment of the invention.

FIG. 2 is a cross-sectional view of a primary screw member of the compression screw assembly.

FIG. 3 is a cross-sectional view of a screw head of the compression screw assembly, which was shown in FIG. 1.

FIG. 4 is a perspective view of the compression screw driver assembly of the orthopedic fixation system, which was shown in FIG. 1.

FIG. 5A is a partially unassembled perspective view of the component of the compression screw driver assembly, which was shown in FIG. 4.

FIG. 5B is a perspective view of a proximal compression shaft member of the compression screw driver assembly of the preferred embodiment.

FIG. 5C is a partial cross-sectional perspective view of the clutch assembly of the compression screw driver assembly according to the preferred embodiment of the invention.

FIG. 5D is an exploded perspective view of the distal compression shaft member of the compression screw driver assembly coupled to the compression driver of the compression screw driver assembly.

FIG. 5E is an exploded perspective view of the primary shaft member of the compression screw driver assembly coupled to the primary driver of the compression screw driver assembly.

FIG. 6A is a front view of the compression screw assembly shown in FIG. 1 having a guide wire and being inserted into bone fragments.

FIG. 6B is partial and transparent perspective view of the compression screw driver assembly shown in FIGS. 1 and 4, but with the compression screw driver assembly positioned in a locked mode.

FIG. 6C is a front view of the compression screw assembly shown in FIGS. 1 and 6A, but with the compression screw assembly coupled to bone fragments.

FIG. 6D is a partial and transparent perspective view of the compression screw driver shown in FIGS. 1 and 4, but with the compression screw driver in an unlocked mode.

FIG. 6E is a front view of the compression screw assembly shown in FIGS. 1, 6A and 6C but with the compression screw assembly applying a compressive force to draw bone fragments to each other.

FIG. 7 is a flow chart, which illustrates the method of coupling the compression screw assembly, shown in FIGS. 1-6E, to a bone fracture joint.

### DETAILED DESCRIPTION OF THE INVENTION

The invention may be understood more readily by reference to the following detailed description of preferred embodiment of the invention. However, techniques, systems and operating structures in accordance with the invention may be embodied in a wide variety of forms and modes, some of which may be different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

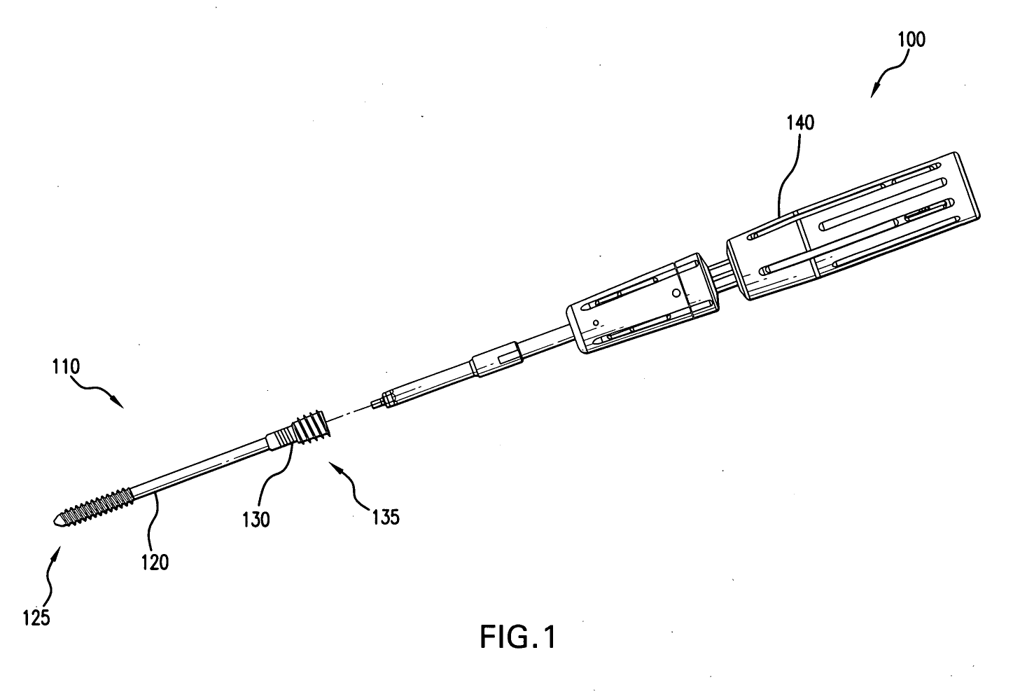

Referring now to FIG. 1, there is shown an orthopedic fixation system 100 which is made in accordance with the teachings of the preferred embodiment of the invention. As shown, the orthopedic fixation system 100 includes a compression screw assembly 110, comprising a primary screw member 120 coupled to a screw head 130. Primary screw member 120 is provided on proximal end 125 of compression screw assembly 110 while screw head 130 is provided on the distal end 135 of the compression screw assembly 110. Primary screw member 120 is generally planar and is coupled to screw head 130 along the same longitudinal axis.

In addition, orthopedic fixation system 100 includes compression screw driver assembly 140 (also called screw driver assembly 140) utilized to couple compression screw assembly 110 to fragmented bones (not shown). Compression screw driver assembly 140 may be utilized to independently apply torque to either primary screw member 120 or screw head 130, although, in other non-limiting examples, compression screw driver assembly 140 may be utilized to apply torque to primary screw member 120 and screw head 130 at the same time. It should be appreciated that in one non-limiting embodiment, the compression screw assembly 110 may be made from Titanium, although, in other non-limiting embodiments, compressive screw assembly 110 may be made from Stainless Steel (SST), Polyetheretherketone (PEEK), Nitinol (NiTi), Cobalt Chrome or other similar types of materials. It should also be appreciated that compression screw assembly 110 is intended for fixation of intra-articular and extra-articular fractures and non-unions of small bones and small bone fragments, arthrodesis of small joints, bunionectomies and osteotomies, such as but not limited to tarsals, metatarsals, carpals, metacarpals, radial head, radial styloid and scaphoid.

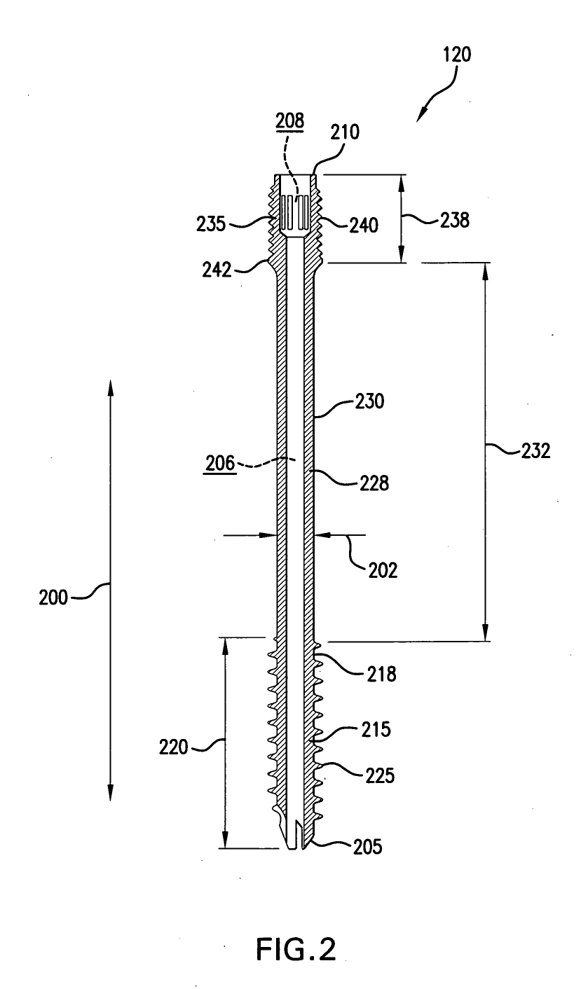

As shown in FIG. 2, primary screw member 120 is generally tubular in shape and has a uniform diameter 202 from tapered end 205 to open end 210. Also, primary screw member 120 has an internal aperture 206 that traverses longitudinal axis 200 and terminates into a generally hexagonal torque-transmitting aperture 208. Apertures 206 and 208 cooperate to form a continuous opening (or cannula) that longitudinally traverse primary screw member 120 from tapered end 205 to open end 210 (i.e., primary screw member 120 is cannulated). The continuous opening or cannula is provided to interact with a guide wire (not shown) by receiving the guide wire within the continuous opening thereby in order to position and locate the primary screw member 120 on bone.

Also shown, primary screw member 120 has a first leading portion 215 having a length 220 and a plurality of symmetrical trapezoidal threads, such as threads 225. Threads 225 are circumferentially disposed on external surface 218 of leading portion 220. Threads 225 have a pitch P1 (i.e., the distance from one point on a screw thread to a corresponding point on the next screw thread, measured parallel to the longitudinal axis 200 of primary screw member 120) and screw lead L1 (i.e., distance the thread 225 advances on one rotational turn of the primary screw member 120). First leading portion 215 may also be provided with a plurality of self-tapping and self-drilling leading edges, such as tapered end 205. Tapered end 205 operates to cause first leading portion 215 to remove bone material when primary screw member 120 is inserted into bone.

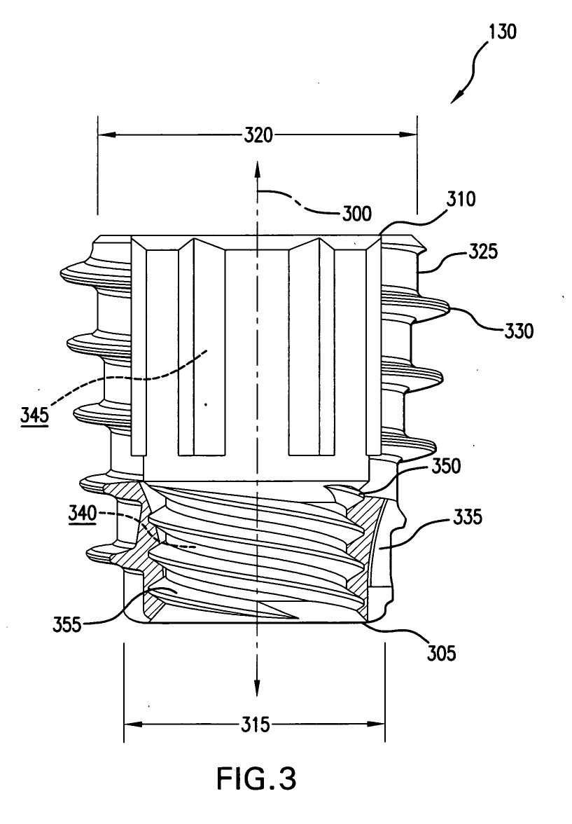

Also as shown, leading portion 215 terminates into a middle portion 228. Particularly, middle portion 228 has a smooth exterior surface 230 for length 232 and terminates into trailing portion 235. Trailing portion 235 has a length 238 and a plurality of symmetrical external threads, such as external threads 240, which are circumferentially disposed on the external surface 242 of portion 235. External threads 240 are machine formed and have a pitch P2 and screw lead L2. Pitch P2 is dissimilar to pitch P1 of circumferential threads 225 on leading portion 215, with pitch P1 being greater than pitch P2. Furthermore, screw lead L2 is dissimilar to screw lead L1. In the preferred embodiment, P1 is three times the pitch of P2, although in other non-limiting embodiments, screw lead L2 is four times pitch P2. In still other embodiments, the pitch differential of P1 and P2 could be more or less. Trailing portion 235 is coupled to compression screw member by threadably coupling external threads 240 to trapezoidal threads 350 (shown in FIG. 3) thereby coupling screw head 130 to primary screw member 120. It should be appreciated that primary screw member 120 has a swaged or disruptive thread located on external threads 240. This swaged thread blocks disassembly of the primary screw member 120 from screw head 130.

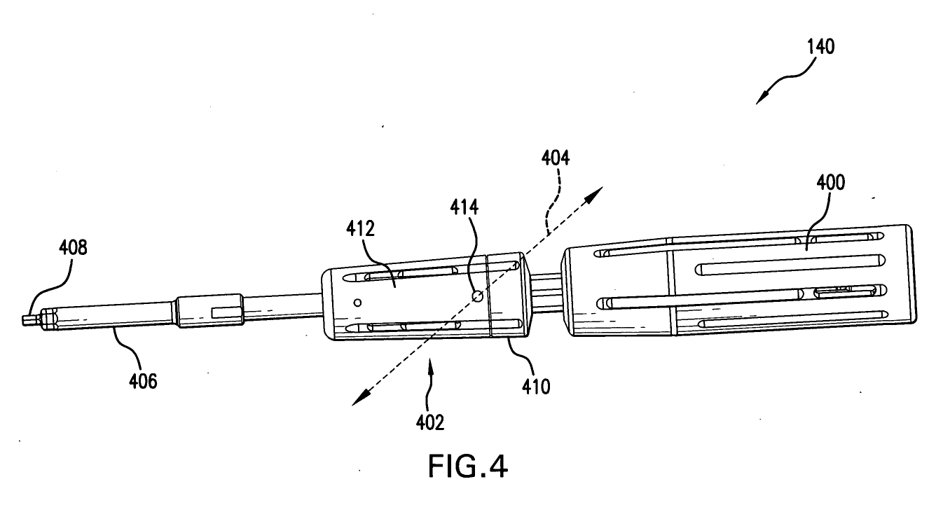

Also as shown, trailing portion 235 has a generally hexagonal torque transmitting aperture 208 formed inside portion 235, with aperture 208 terminating into open end 210. Torque transmitting aperture 208 is provided to receive a complementary hexagonal-shaped drive tip 408 (shown in FIG. 4) so that torque is selectively transferred from compression screw driver assembly 140 to compression screw assembly 110 when compression screw driver assembly 140 is received in aperture 208 and screw head 130 and subsequently rotated in any arcuate direction that causes primary screw member 120 to rotate. It should be appreciated that in other non-limiting embodiments, a star-shaped aperture, a square-shaped aperture, or any other shaped aperture may be utilized without departing from the scope of the invention. It should also be appreciated that the length of primary screw member 120 may be selected of varying lengths to allow a surgeon to fuse different size bone fragments together, such as, for example, the scaphoid, foot and ankle bones.

Referring now to FIG. 3, screw head 130 is generally frustoconical 305 shape and tapers from first end 310 to second end 305 (i.e., internal diameter 315 is smaller than internal diameter 320). Screw head 130 has a plurality of symmetrically circular threads, such as threads 330, which are circumferentially disposed on external surface 325. Circular threads 330 being formed on tapered surface 325 subsequently cause circular threads 330 to taper as well. Circular threads 330 have a pitch P3 and screw lead L3, which is substantially the same as pitch P1 and screw lead L1 of circumferential threads 225 on first leading portion 215. Screw head 130 may also be provided with a plurality of self-tapping leading edges, such as self-tapping leading edge 335. Self-tapping leading edge 335 operates to cause screw head 130 to remove bone material when primary screw member 120 and screw head 130 are coupled to each other and inserted into bone (not shown).

Also shown, screw head 130 contains a generally hexagonal torque-transmitting aperture 345, which terminates into circular aperture 340. Circular aperture 340 has an internal diameter that is substantially the same as the external diameter of trailing portion 235 so as to securely and threadably couple primary screw member 120 with screw head 130. Also, torque-transmitting aperture 345 is provided to receive a complementary hexagonal-shaped drive tip 406 (shown in FIG. 4) so that torque is transferred from screw driver assembly 140 to screw head 130 when compression screw driver assembly 140 is received in torque-transmitting aperture 345 at end 310 and subsequently rotated in any arcuate direction that causes screw head 130 to rotate. It should be appreciated that in other non-limiting embodiments, a star-shaped aperture, a square-shaped aperture, or any other shaped aperture may be utilized without departing from the scope of the invention. Apertures 340 and 345 are aligned along vertical axis 300 and cooperate to form a continuous opening or cannula that longitudinally traverse screw head 130 from end 310 to end 305 (i.e., screw head 130 is cannulated). The continuous opening or cannula is provided to interact with a guide wire (not shown) by receiving the guide wire within the continuous opening thereby assisting in the positioning and locating of screw head 130. Further, screw head 130 has a plurality of trapezoidal threads 350 formed on internal surface 355. Trapezoidal threads 350 are complementary to external threads 240 of primary screw member 120 (i.e., trapezoidal threads 350 have pitch P4 and screw lead L4, which is substantially the same as pitch P2 and screw lead L2). Screw head 130 receives primary screw member 120 by receiving trailing portion 235 of screw head 130 within aperture 340. In this manner, external threads 240 of primary screw member 120 are threadably coupled to trapezoidal threads 350 of screw head 130. In this configuration, the difference in screw leads L3 and L4 causes a compressive force or compression to be applied by external threads 330 on bone fragments when primary screw member 120 and screw head 130 are coupled to each other and inserted into bone.

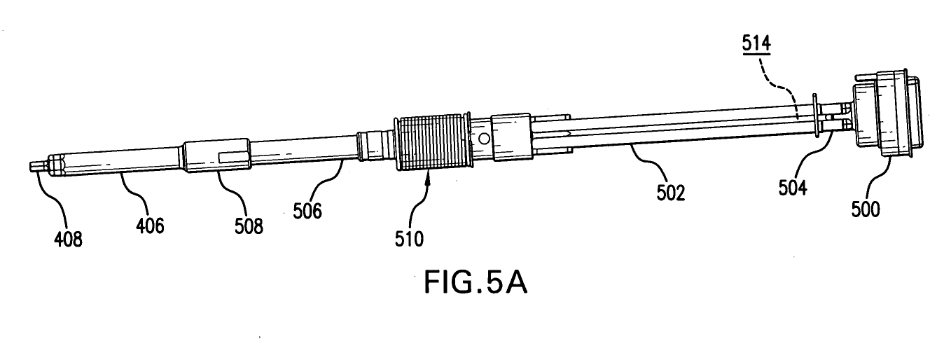

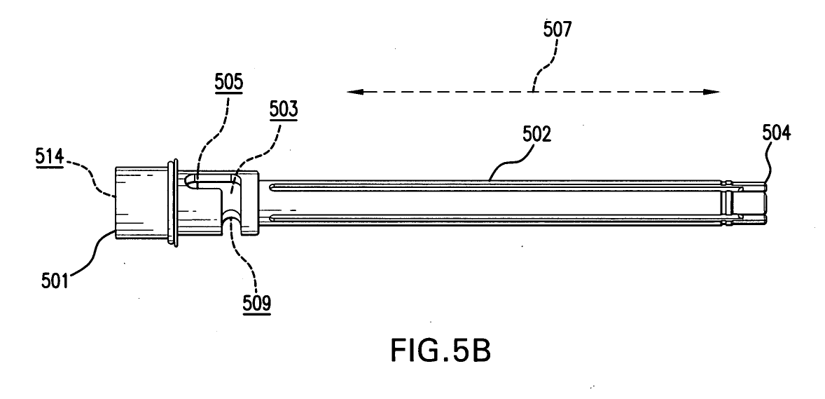

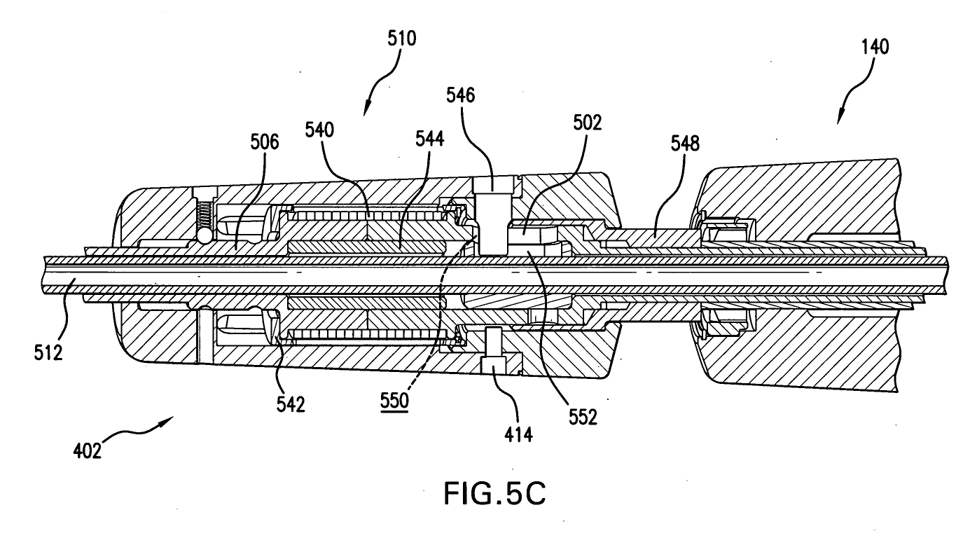

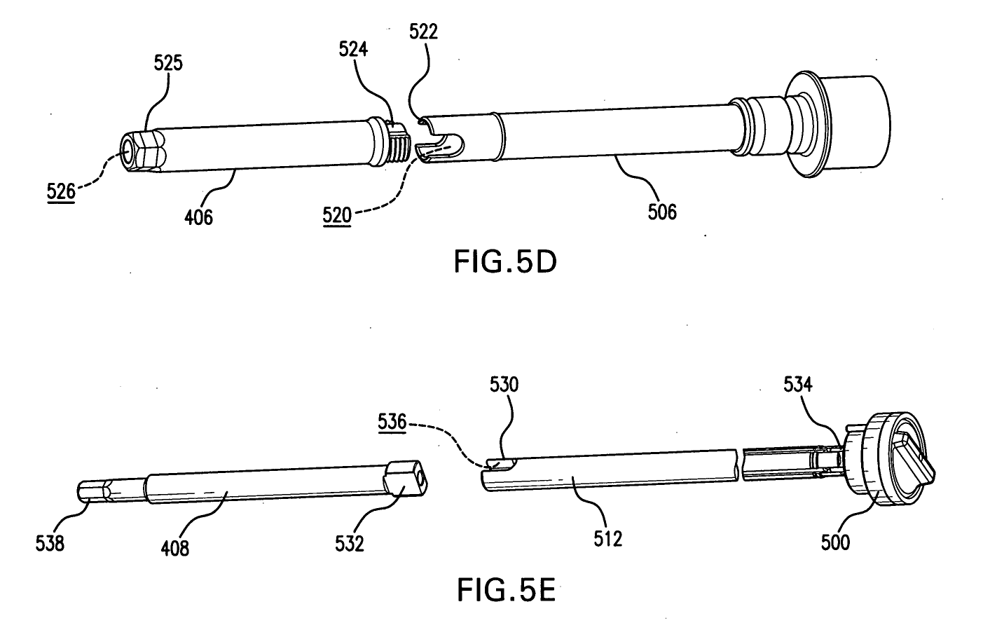

Referring now to FIG. 4, compression screw driver assembly 140 is illustrated for coupling compression screw assembly 110 to bone fragments. Particularly, compression screw driver assembly 140 includes a main handle portion 400 enclosing a ratchet assembly 500 (shown in FIG. 5A). Handle portion 400 is slidably coupled to a collar assembly 402, which terminates into a plurality of hexagonal drive tips 406 and 408. Hexagonal drive tip 406 is utilized for inserting screw head 130 while hexagonal drive tip 408 is utilized for inserting primary screw member 120. Collar assembly 402 comprises a first generally cylindrical shaped proximal collar 410 coupled to a second generally cylindrical-shaped distal collar 412, with the collars 410 and 412 coupled together through a pin 414. Pin 414 traverses a through-aperture (not shown) formed in collar 410 aligned on an orthogonal axis 404 on proximal collar 410 and also traverses a through aperture on distal collar 412 which is aligned on the same orthogonal axis 404 (i.e., apertures form 90-degree angle to external surfaces of collars 410 and 412), thereby securely and frictionally coupling proximal collar 410 to distal collar 412.

As shown in FIG. 5A, ratchet assembly 500 is slidably coupled to proximal shaft member 502 at first end 504. It should be appreciated that ratchet assembly 500 includes features that are generally known in the art in order to cause the proximal shaft member 502 to rotate either clockwise or counter clockwise by adjusting ratchet assembly 500 in a corresponding direction. Also, proximal shaft member 502 is generally tubular and encloses a longitudinally coextensive cavity (not shown), which is provided to receive primary shaft member 512 (not shown in FIG. 5A; shown in FIG. 5C). Primary shaft member 512 abuts ratchet assembly 500 at a first end, resides within longitudinal cavities of proximal shaft member 502 and distal shaft member 506 and terminates within the enclosed cavity of distal collar 508. Proximal shaft member 502 is coupled to distal shaft member 506 within a clutch assembly 510. Clutch assembly 510 controls the rotation of hexagonal drive tips 406 and 408 relative to each other, which will be described below. Also shown, clutch assembly 510 couples proximal shaft member 502 to primary shaft member 512 (shown in FIG. 5C).

Additionally and as shown in FIG. 5B, proximal shaft member 502 includes a generally “L-shaped” groove 503 residing at end 501, which is directly opposite end 504. Groove 503 has a first horizontal slot 505 (i.e., slot 505 is along the longitudinal axis 507) that terminates into an orthogonal slot 509 (i.e., slot 509 forms a 90-degree angle with slot 505). L-shaped groove 503 is provided to receive pin 546 (shown in FIG. 5C) to cause compression screw driver assembly 140 (not shown) to selectively engage screw head 130 (not shown) and primary screw member 120 (not shown), thereby selectively imparting torque on screw head 130 (not shown) and primary screw member 120 (not shown) when compression screw driver assembly 140 is selectively rotated. It should be appreciated that slot 505 is the longitudinal slot being utilized for insertion of both primary screw member 120 (not shown) and screw head 130 (not shown) while slot 509 is the radial slot and controls screw head 130 (not shown) only.

As shown in FIG. 5C, collar assembly 402 of compression screw driver assembly 140 encloses clutch assembly 510, which is operably coupled to primary shaft member 512 and proximal shaft member 502. Particularly, clutch assembly 510 comprises a one-way spring member 540 circumferentially enclosing external surfaces of proximal shaft member 502 and distal shaft member 506. Spring member 540 is provided to permit constrained relative motion of shaft members 502 and 512. Spring member 540 is securely coupled to proximal shaft member 502 and also to distal shaft member 506 through a generally cylindrical retainer clutch member 542. Spring member 540 applies a “compressive force” on members 502 and 506 such as the force applied by compressing a spring. Also, proximal shaft member 502 and distal shaft member 506 encloses primary shaft member 512 and are separated by a bearing sleeve 544.

Also shown, a generally tubular spline 548 is provided which receives proximal shaft member 502 and primary shaft member 512. In addition, compression screw driver assembly 140 includes a pin member 546 that operably couples clutch assembly 510 to primary shaft member 512 and proximal shaft member 502. Pin 546 is received in orthogonal aperture 550 of proximal shaft member 502 as well as being received in aperture (not shown) of primary sleeve 552. The aligned apertures of proximal shaft member 502 and primary sleeve 552 selectively causes pin member 546 to engage proximal shaft member 510 as well as primary shaft member 512 and causes compression screw driver assembly 140 to have a plurality of mechanical modes interchangeable by pin member 546. A user may utilize this compression screw driver assembly 140 to either transmit insertion torque to the entire compression screw assembly 110 or allow for the controlled application of compression to the screw head 130. These modes are shown and described below.

As shown in FIG. 5D, proximal shaft member 502 terminates into a tubular distal shaft member 506. Distal shaft member 506 is coupled to hexagonal drive tip 406 through an interference fit within the internal cavity of distal collar 508 (not shown). Particularly, distal shaft member 506 is tubular (i.e., distal shaft member 506 encloses a longitudinally coextensive cavity 520) and terminates into a generally “U-shaped” end 522. End 522 receives a complementary shaped threaded end 524 of tubular hexagonal drive tip 406 with hexagonal drive tip 406 having a hexagonal shaped end 525. Hexagonal shaped end 525 is received within a complementary torque-transmitting aperture 345 (shown in FIG. 3) of screw head 130. In other non-limiting embodiments, distal shaft member 506 may be coupled to hexagonal drive tip 406 through a screw, pin or other similar types of attachment techniques. Hexagonal drive tip 406, being tubular, has a longitudinally coextensive cavity 526, which is provided to receive hexagonal drive tip 408. Distal collar 508 reinforces the connection and prevents the distal shaft member 506 from separating (i.e., sliding out of contact with hexagonal drive tip 406). In operation, ratchet assembly 500 (shown in FIG. 5A) drives proximal shaft member 502 (i.e., locks the proximal shaft member 502 in position). In this position, proximal shaft member 502 may be rotated either clockwise or counterclockwise by rotating handle portion 400 (shown in FIG. 4) in a corresponding direction, which causes torque to be transferred through distal shaft member 506 and to hexagonal drive tip 406.

As shown in FIG. 5E, primary shaft member 512 abuts ratchet assembly 500 at a first end 534 and terminates into a generally “U-shaped” end 530, with end 520 being substantially similar to generally “C-shaped” end 522 of distal shaft member 506. End 530 receives a generally rectangular end 532 of hexagonal drive tip 408 within the plurality of grooves, such as groove 536. In other non-limiting embodiments, primary shaft member 512 may be coupled to hexagonal drive tip 408 through a screw, pin or other similar types of attachment techniques. Hexagonal drive tip 408 also has a hexagonal shaped end 538, which is provided to be received in primary screw member 120 (shown in FIG. 2). Primary shaft member 512 and hexagonal drive tip 408 are generally tubular (i.e., cannulated) and receive a “guide wire” (also called Kirschner wire).

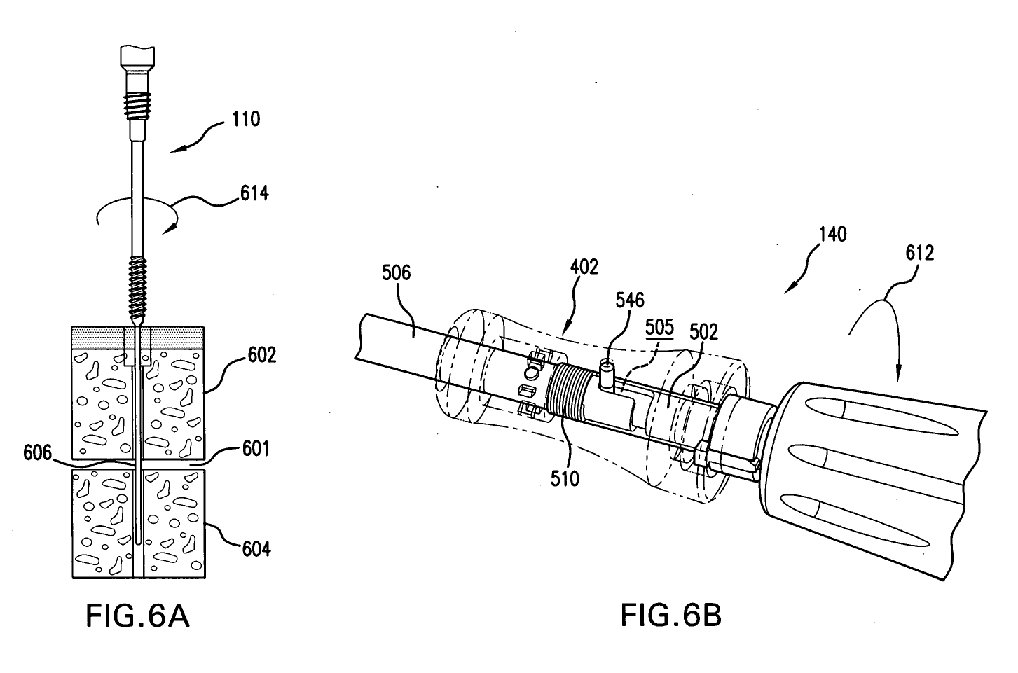

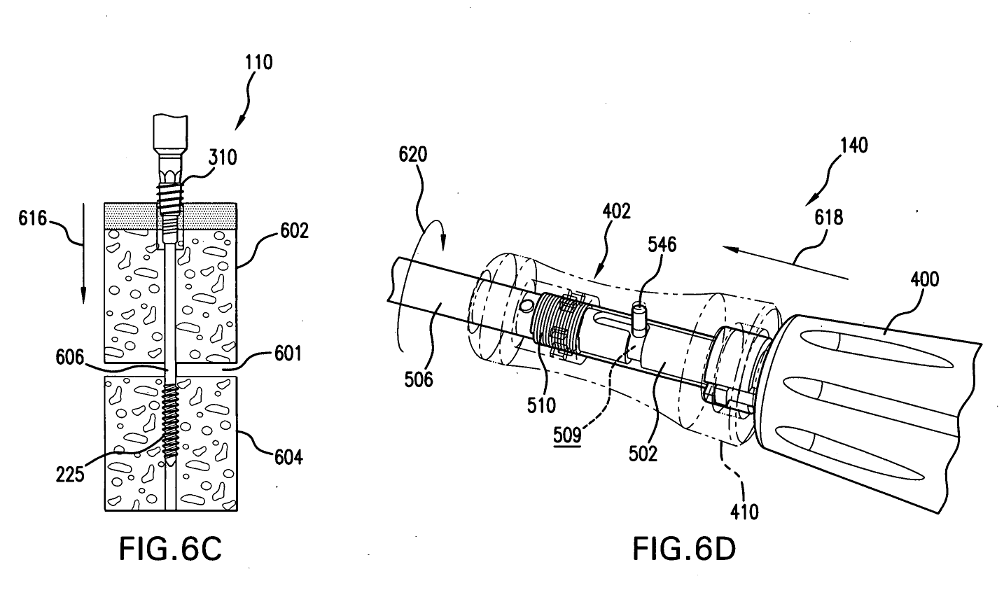

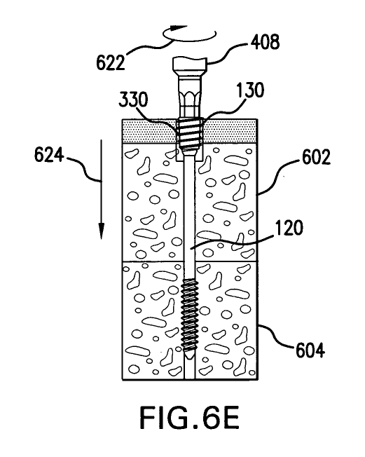

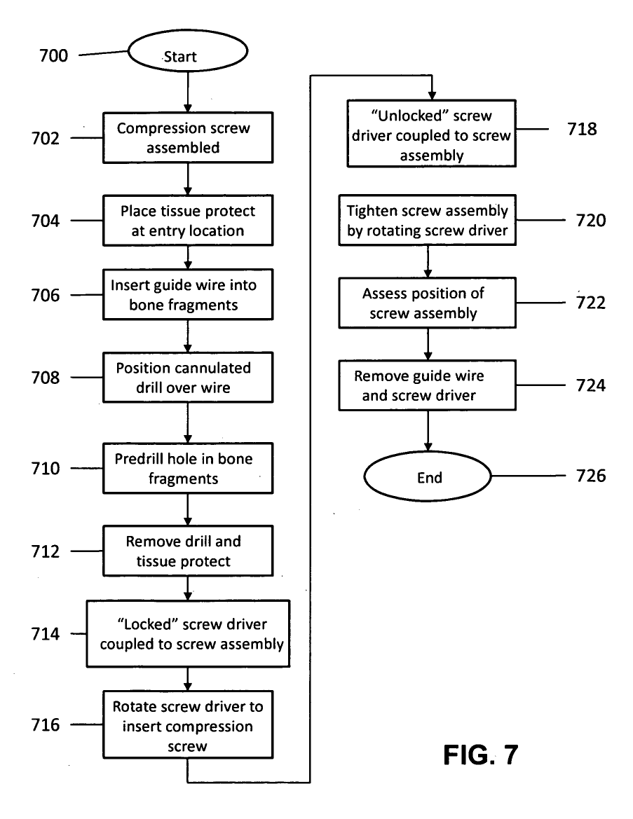

In operation, and as best shown in FIGS. 6A-7, orthopedic fixation system 100, comprising compression screw assembly 110 and compression screw driver assembly 140 (not shown), may be utilized to provide a system for individually applying compression to separated bone fragments across a fracture site. Compression screw assembly may be selectively assembled, as was shown in FIGS. 1, 2 and 3. Particularly, trailing portion 235 of primary screw member 120 is inserted into circular aperture 340 of screw head 130 and trailing portion 235 is rotated until external threads 240 engage trapezoidal threads 350. This rotation causes portion 235 to travel into circular aperture 340. In other non-limiting embodiments, compression screw assembly 110 may be provided to a user, for example a surgeon, in an assembled condition.

Next and as shown in FIG. 6A, compression screw assembly 110 may be inserted through a plurality of bone fragments 602 and 604, with bone fragments 602 and 604 being located on opposed ends of bone fracture site 601. Compression screw assembly 110 may be selectively positioned inside bone fragments 602 and 604 by placing a tissue protector or guide (not shown) at entry location of compression screw assembly 110 and a guide wire 606 is drilled (FIG. 6A) through bone segments 602 and 604 to correct depth and placement. Next, a cannulated drill is positioned over exposed end of guide wire 606 and inside tissue protector or guide (not shown). Guide wire 606 serves as an anchoring system for the cannulated drill guide and resists migration of the cannulated drill during drilling. Next, a hole is predrilled to the correct depth within bone fragments 602 and 604. The cannulated drill travels along the path of guide wire 606 as guide wire 606 is received within the longitudinal cavity of the drill. Next, the tissue protector or guide, and drill are removed and a cannulated counter sink (not shown) is placed over guide wire 606 and counter-sinked to an appropriate depth and removed.

Next, as shown in FIGS. 6A and 6B, compression screw assembly 110 is coupled to compression screw driver assembly 140 in the locked position (i.e., the “screw insertion mode”) and placed over the wire guide 606. Next, as shown in FIGS. 6B and 6C, compression screw driver assembly 140, in the “screw insertion” mode, is rotated in a clockwise direction 612. Particularly, as shown in FIG. 6B, pin 546 within collar assembly 402 is positioned in aperture 505, which causes proximal shaft member 502 and distal shaft member 506 to be locked together. Pin 546 also causes primary shaft member 512 (not shown in FIGS. 6B-6C; shown in FIG. 5C) to be engaged. In this position, clutch assembly 510 (shown in FIG. 6B) operates “normally,” whereby clockwise rotation of handle portion 400 along arc 612 causes the proximal shaft member 502, distal shaft member 506 and primary shaft member 512 (shown in FIG. 5C) to rotate in a respective clockwise direction along same arc 612. Thus, as shown in FIG. 6C, compression screw driver assembly 140 causes the hexagonal drive tips 406 and 408 to rotate together along direction of arc 612 driving both primary screw member 120 and screw head 130 (i.e., there is no relative rotations inside collar assembly 402 while ratchet assembly 500 operates for ratcheting action). Rotating compression screw assembly 110 causes compression screw assembly 110 to travel into bone segments 602 and 604 and across bone fracture 601, while guide wire 606 (shown in FIG. 6A) is pulled gently in order to feed guide wire 606 (shown in FIG. 6A) through compression screw assembly 110. It should be appreciated that plurality of circumferential threads, such as threads 225 and 330 on compression screw assembly 110, causes the plurality of circumferential threads 225 and 330 to grip or catch the bone segments 602 and 604. This causes the compression screw assembly 110 to travel into bone segments 602 and 604 (shown in FIG. 6C) in direction 616 as compression screw assembly 110 is rotated. Compression screw assembly 110 is inserted into bone segments 602 and 604 until end 310 of screw head 130 is flush with the external surface of bone 602 (i.e., counter-sinking screw head 130).

Next, and as shown in FIG. 6D, compression screw driver assembly 140 is positioned in the unlocked position (i.e., the “compression mode”). In the “compression mode”, pin 546 resides within collar assembly 402 and is positioned in aperture 509. This causes clutch assembly 510 to engage primary shaft member 512 only (shown in FIG. 5C). A user would grasp collar assembly 402 while driving handle portion 400. This will cause handle portion 400 to travel towards collar assembly 402 in direction 618 causing proximal collar 410 to abut handle portion 400. As handle portion 400 is rotated clockwise in arcuate direction 620, the proximal shaft member 502 sweeps radially within slot 509 causing the distal shaft member 506 to sweep radially (i.e., back and forth) through slot 509 and consequently rotates screw head 130 (shown in FIG. 6E) without rotating primary screw member 120 (shown in FIG. 6E). Therefore, primary shaft member 512 is held fixed while the distal shaft member 506 transmits torque to the hexagonal drive tip 408 causing torque to be transmitted to screw head 130. In this mode, hexagonal drive tip 408 applies a counter-torque on primary screw member 120 and prevents it from rotating as screw head 130 is rotated.

Next, as shown in FIG. 6E, screw head 130 is further rotated in a clockwise direction 622. Rotating screw head 130 causes screw head 130 to further travel into bone segment 602. The difference in screw lead L3 on external thread 330 and screw lead L4 on screw head 130 (which is the same as screw lead L4 on primary screw member 120) causes a compressive force or compression to be applied by external threads 330 on bone fragments 602 and 604 when screw head 130 is inserted into bone. The larger screw leads L4 of screw head 130 relative to external thread leads L3 causes primary screw member 120 to be drawn towards screw head 130 causing bone fragments 602 and 604 to be drawn together. This applies a compressive force to separated bone fragments 602 and 604. It should be appreciated that plurality of circumferential threads, such as threads 330, are provided so that rotating screw assembly 110 causes the plurality of threads 330 to grip or catch the bone segment 602 and causes the screw head 130 to travel into bone segment 602 in direction 624.

Next, the position of the compression screw assembly 110 is assessed and if required, compression screw driver assembly 140 may be switched to the locked position (i.e., “screw insertion mode”) to change the depth of the compression screw assembly 110. Next, guide wire 606 and compression screw driver assembly 140 are removed. It should be appreciated that compression screw driver assembly 140 may be also be utilized for removal of compression screw assembly 110 from bone fragments 602 and 604 (shown in FIG. 6E) by controlling rotation of hexagonal drive tip 408 (shown in FIG. 4), causing the primary screw member 120 to rotate in a direction that retracts the primary screw member 120 from bone.

Referring now to FIG. 7, there is shown a flow chart for utilizing orthopedic fixation system 100 to insert compression screw assembly 110 in bone. The method starts in step 700 and proceeds to step 702, whereby compression screw assembly 110 may be selectively assembled. Next, in step 704, tissue protect or guide is placed at the entry location of compression screw assembly 110 and, in step 706, a guide wire is drilled through bone fragments (shown in FIG. 6A). Next, in step 708, a cannulated drill is positioned over the exposed end of guide wire and inside tissue protector or guide. Next, in step 710, a hole is predrilled to the correct depth within bone fragments 602 and 604 (shown in FIG. 6A). Next, in step 712, the tissue protect or guide, and cannulated drill are removed and a cannulated counter sink is placed over guide wire, counter-sinked to an appropriate depth, and removed.

Next, in step 714, compression screw assembly 110 is coupled to compression screw driver assembly 140 in the locked position (i.e., the “screw insertion mode”) and placed over the guide wire 606. Next, in step 716, compression screw driver assembly 140, while in the “screw insertion” mode, is rotated in order to rotate compression screw assembly 110 and correspondingly insert into bone fragments 602 and 604 (shown in FIG. 6C). Next, in step 718, compression screw driver assembly 140 is positioned in the unlocked position (i.e., the “compression mode”) and causing screw head 130 (shown in FIG. 6C) to rotate while preventing primary screw member 120 (shown in FIG. 6C) to rotate. Next, in step 720, screw head 130 is further rotated causing screw head 130 to travel into bone segments 602 and 604 (shown in FIG. 6E) and drawing primary screw member 120 towards screw head 130 (shown in FIG. 6E). Next, in step 722, the position of the compression screw assembly 110 is assessed and if required, compression screw driver assembly 140 may be switched to the locked position to adjust the depth of the compression screw assembly 110. Next, in step 724, guide wire 606 and compression screw driver assembly 140 are removed. The method ends in step 726.

It should be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

What is claimed is:

1. A compression screw apparatus comprising:
a primary screw member having a threaded leading portion, an opposite threaded trailing portion and a smooth middle portion disposed between said leading portion and said trailing portion, said leading portion having a plurality of first threads having a first pitch and said trailing portion having a plurality of second threads having a second pitch; and
a screw head having an outer threaded surface, said outer threaded surface having a plurality of third threads having a third pitch, and wherein said screw head defines a central opening with a threaded inner surface, said threaded inner surface having a plurality of threads having a fourth pitch, wherein said threaded inner surface is adapted for mating engagement on said threaded trailing portion of said primary screw member;
wherein said first pitch and said third pitch are approximately identical, and wherein said second pitch and said fourth pitch are approximately identical; and
wherein said screw leads of said threaded trailing portion are greater than said second pitch.

2. The compression screw apparatus of claim 1 wherein said primary screw member is cannulated.

3. The compression screw apparatus of claim 1 wherein said smooth middle portion of said primary screw member has an unthreaded transition portion between said leading portion and said trailing portion of said primary screw.

4. The compression screw apparatus of claim 1 wherein said threads of said leading portion are of a greater pitch than said threads of said trailing portion.

5. The compression screw apparatus of claim 1 wherein said screw leads of said trailing portion are at least three times said second pitch.

6. The compression screw apparatus of claim 1 wherein the root of said first threads of said leading portion is deeper than the root of said second threads of said trailing portion.

7. The compression screw apparatus of claim 1 wherein said third plurality of threads of said screw head are tapered toward said trailing end of said primary screw member.

8. The compression screw apparatus of claim 7 wherein said central opening of said screw head has a diameter that decreases toward an end of said screw head adapted for first advancing onto said trailing portion of said primary screw member.

9. The compression screw apparatus of claim 1 wherein said second plurality of threads on said trailing end are chamfered to prevent uncoupling of said screw head from said trailing end.

10. The compression screw apparatus of claim 1 wherein the diameter of said outer threads of said screw head is larger than a diameter of said threads of said leading portion of said primary screw member.

11. The compression screw apparatus of claim 1 wherein the pitch of said outer threads of said screw head is approximately identical to the pitch of said threads of said leading portion of said primary screw member.

12. The compression screw apparatus of claim 1 wherein said threads of said inner surface of said screw head has a lead that is at least three time larger than the lead of said threads of said outer surface of said screw head.

13. The compression screw apparatus of claim 1 wherein an end of said central opening is adapted for receiving a driver.

14. The compression screw apparatus of claim 1 wherein an open end of said trailing portion is adapted for receiving a driver.

15. A fixation system comprising:
a compression screw apparatus for compressing bone, said compression screw apparatus comprising:
a primary screw member having a threaded leading portion, an opposite threaded trailing portion and a smooth middle portion disposed between said leading portion and said trailing portion, said leading portion having a plurality of first threads having a first pitch and said trailing portion having a plurality of second threads having a second pitch;
a screw head having an outer threaded surface, said outer threaded surface having a plurality of third threads having a third pitch, and wherein said screw head defines a central opening with a threaded inner surface, said threaded inner surface having a plurality of threads having a fourth pitch, wherein said threaded inner surface is adapted for mating engagement on said threaded trailing portion of said primary screw member;
wherein said first pitch and said third pitch are approximately identical, and wherein said second pitch and said fourth pitch are approximately identical, and wherein said screw leads of said threaded trailing portion are greater than said second pitch; and
a screw driver assembly for engaging said compression screw assembly, said screw driver assembly comprising:
a proximal compression shaft member having a first end and an opposed second end, said first end coupled to a ratchet assembly and said second end receiving a pin for controlling rotation of said screw driver assembly;
a distal compression shaft member having a third end coupled to said second end of said proximal compression shaft member and a fourth end for controlling rotational movement of said compression screw member;
a primary shaft member residing within said proximal shaft member and also residing within said distal shaft member, wherein said primary shaft member having an end which is provided for controlling rotational movement of said primary screw member; and
a clutch assembly for selectively engaging and controlling rotational movement of said compression screw and said primary screw.

16. The fixation system of claim 15 wherein said primary screw member is cannulated.

17. The fixation system of claim 15 wherein said smooth middle portion of said primary screw member has an unthreaded transition portion between said leading portion and said trailing portion of said primary screw.

18. The fixation system of claim 15 wherein said threads of said leading portion are of a greater pitch than said threads of said trailing portion.

19. The fixation system of claim 15 wherein said screw leads of said trailing portion are at least three times said second pitch.

20. The fixation system of claim 15 wherein the root of said first threads of said leading portion is deeper than the root of said second threads of said trailing portion.

21. The fixation system of claim 15 wherein said third plurality of threads of said screw head are tapered toward said trailing end of said primary screw member.

22. The fixation system of claim 21 wherein said central opening of said screw head has a diameter that decreases toward an end of said screw head adapted for first advancing onto said trailing portion of said primary screw member.

23. The fixation system of claim 15 wherein said second plurality of threads on said trailing end are chamfered to prevent uncoupling of said screw head from said trailing end.

24. The fixation system of claim 15 wherein the diameter of said outer threads of said screw head is larger than a diameter of said threads of said leading portion of said primary screw member.

25. The fixation system of claim 15 wherein the pitch of said outer threads of said screw head is approximately identical to the pitch of said threads of said leading portion of said primary screw member.

26. The fixation system of claim 15 wherein said threads of said inner surface of said screw head has a lead that is at least three time larger than the lead of said threads of said outer surface of said screw head.

27. The fixation system of claim 15 wherein an end of said central opening is adapted for receiving a driver.

28. The fixation system of claim 15 wherein an open end of said trailing portion is adapted for receiving a driver.

29. A method of compressing bone fragments, the method comprising the steps of:
providing a compression screw assembly;
placing a guide at an entry location of a compression screw assembly into bone;
inserting a guide wire into the bone at the entry location;
drilling a hole in the entry location to a predetermined depth;
coupling the compression screw assembly to a screw driver assembly;
rotating the compression screw driver assembly to insert compression screw assembly into bone;
rotating the compression screw driver assembly to compress bone fragments.

30. The method of claim 29, wherein the compression screw assembly further comprises:
a primary screw member having a threaded leading portion, an opposite threaded trailing portion and a smooth middle portion disposed between the leading portion and the trailing portion, the leading portion having a plurality of first threads having a first pitch and the trailing portion having a plurality of second threads having a second pitch; and
a screw head having an outer threaded surface, the outer threaded surface having a plurality of third threads having a third pitch, and wherein the screw head defines a central opening with a threaded inner surface, the threaded inner surface having a plurality of threads having a fourth pitch, wherein the threaded inner surface is adapted for mating engagement on the threaded trailing portion of the primary screw member;
wherein the first pitch and the third pitch are approximately identical, and wherein the second pitch and the fourth pitch are approximately identical; and
wherein the screw leads of the threaded trailing portion are greater than the second pitch.

31. The method of claim 30, wherein the primary screw member is cannulated.

32. The method of claim 31, wherein the smooth middle portion of the primary screw member has an unthreaded transition portion between the leading portion and the trailing portion of the primary screw.
